SEQUENCE LISTING (1 sequence)

SEQ ID NO: 1 - DNA, 17 bp, Unknown
  Description of Unknown Target molecule sequence
tgcataggcgccgttca

(19) United States
(12) Patent Application Publication
(10) Pub. No.: US 2021/0118527 A1
(43) Pub. Date: Apr. 22, 2021

(54) Using Machine Learning to Optimize Assays for Single Cell Targeted DNA Sequencing

(71) Applicant: Mission Bio, Inc., South San Francisco, CA (US)

(72) Inventors: Dongmyunghee Kim, South San Francisco, CA (US); Manimozhi Manivannan, South San Francisco, CA (US); Saurabh Gulati, South San Francisco, CA (US); Shu Wang, South San Francisco, CA (US)

(21) Appl. No.: 16/936,378

(22) Filed: Jul. 22, 2020

Related U.S. Application Data
(60) Provisional application No. 62/877,263, filed on Jul. 22, 2019.

(51) Int. Cl. G16B 40/00; G16B 30/00

(52) U.S. Cl. CPC G16B 40/00; G16B 30/00

(57) ABSTRACT

The disclosure generally relates to using machine learning to optimize assays for single cell targeted DNA sequencing. In an exemplary embodiment, amplicons are designed for disease detection assays. An exemplary amplicon design step includes the steps of (1) receiving empirical data of a plurality of initial attributes from a panel of primary amplicons sequenced with target molecules, each of the initial attributes defining at least one performance criteria for a respective amplicon; (2) ranking performance of each amplicon according to a predefined criteria; (3) from among the ranked amplicons, (i) selecting a plurality of key attributes, and (ii) selecting one or more substantially independent and non-correlating attributes, to form a group of selected primary amplicon attributes; (4) calculate a plurality of statistical parameters for each of the selected primary amplicon attributes; and (5) configure a plurality of secondary amplicons wherein the secondary amplicons include secondary amplicon parameters consistent with the statistical parameters of the selected primary amplicons.

The instant disclosure claims priority to the Provisional Application No. 62/877,263 filed Jul. 22, 2019; the disclosure of which is incorporated herein in its entirety.

### SEQUENCE LISTING

The instant application contains a Sequence Listing which has been submitted electronically in ASCII format and is hereby incorporated by reference in its entirety. Said ASCII copy, created on Dec. 14, 2020, is named MSB-016US_SL.txt and is 528 bytes in size.

### FIELD

The instant disclosure generally relates to methods, apparatus and systems for using machine learning to optimize assays for single cell targeted DNA analysis.

### BACKGROUND

Assays are conventionally used for qualitatively assessing or quantitatively measuring the presence, amount, or functional activity of a target entity. The target entity, also known as the analyte, may be a DNA or an RNA fragment, a protein, a lipid or any other chemical compound whose presence can be detected. In some applications, assays have been developed to detect presence of a disease by detecting DNA/RNA sequences that correspond to the disease. For example, assays have been developed to detect the presence of multiple myeloma (MM) in patients by detecting DNA fragments (or targets) that correspond to the disease. The timely and accurate detection of MM or other similar tumors is of significant interest to patients and the medical community.

Assay optimization and validation are essential, even when using assays that have been predesigned and commercially obtained. Optimization is implemented to ensure that the assay is as sensitive as is required. Assay optimization is also important to ensure that the assay is specific to the target of interest. For example, pathogen detection or expression profiling of rare mRNAs may require a high degree of sensitivity. Detecting a single nucleotide polymorphism (SNP) requires high specificity. On the other hand, viral quantification needs both high specificity and sensitivity.

Assays requiring high specificity are susceptible if performed without optimization and adequate controls. Further, simultaneous detection of multiple targets in a multiplex reaction requires assay optimization in order to detect and identify all targets.

Assays of high degree of specificity and sensitivity are required for genotyping cell mutations. High throughput single cell DNA sequencing allows for detection of rare mutations in cells and identification of subclones defined by co-occurrence of mutations. This enables researchers to characterize tumor heterogeneity and progression which cannot be achieved by standard bulk sequencing. A significant challenge with multiplex sequencing at single cell level is the non-uniform amplification of the targeted regions during PCR. The non-uniform amplification results in inadequate coverage of mutations of interest in the panel and hence makes genotyping challenging. Thus, there is a need for an automated assay design to provide high accuracy target detection in a multiplexed panel.

### BRIEF DESCRIPTION OF THE DRAWINGS

The disclosed embodiments are discussed with reference to the following exemplary and non-limiting illustrations, in which like elements are numbered similarly, and where:

FIG. 1 is a representation of a single-stranded DNA sequence (SEQ ID NO: 1) of a target molecule;

FIG. 2 illustrates an exemplary flow diagram of an overall ML training process according to one embodiment of the disclosure;

FIG. 3 illustrates an exemplary feature selection algorithm according to one embodiment of the disclosure;

FIG. 4 is an exemplary illustration of a process flow for implementing statistical analysis and the design steps according to one embodiment of the disclosure; and

FIG. 5 shows an exemplary system for implementing an embodiment of the disclosure.

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes the sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further include any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acids and the elongation or extension reaction of this formed nucleic acid occur continuously, a series of these reactions is comprehensively called amplification. The polynucleic acid produced by the amplification technology employed is generically referred to as an “amplicon” or “amplification product.”

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

A ‘barcode’ nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

A barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri™ beads to tag and then later identify each droplet's amplicon content. The use of barcodes is described in U.S. patent application Ser. No. 15/940,850 filed Mar. 29, 2018 by Abate, A. et al., entitled ‘Sequencing of Nucleic Acids via Barcoding in Discrete Entities’, incorporated by reference herein.

In some embodiments, it may be advantageous to introduce barcodes into discrete entities, e.g., microdroplets, on the surface of a bead, such as a solid polymer bead or a hydrogel bead. These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of the complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may be utilized, such as a PCR-based assay, e.g., quantitative PCR (qPCR), or an isothermal amplification may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

The number of amplification/PCR primers that may be added to a microdroplet may vary. The number of amplification or PCR primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

One or both primer of a primer set may also be attached or conjugated to an affinity reagent that may comprise anything that binds to a target molecule or moiety. Non limiting examples of affinity reagent include ligands, receptors, antibodies and binding fragments thereof, peptide, nucleic acid, and fusions of the preceding and other small molecule that specifically binds to a larger target molecule in order to identify, track, capture, or influence its activity. Affinity reagents may also be attached to solid supports, beads, discrete entities, or the like, and are still referenced as affinity reagents herein.

One or both primers of a primer set may comprise a barcode sequence described herein. In some embodiments, individual cells, for example, are isolated in discrete entities, e.g., droplets. These cells may be lysed and their nucleic acids barcoded. This process can be performed on a large number of single cells in discrete entities with unique barcode sequences enabling subsequent deconvolution of mixed sequence reads by barcode to obtain single cell information. This approach provides a way to group together nucleic acids originating from large numbers of single cells. Additionally, affinity reagents such as antibodies can be conjugated with nucleic acid labels, e.g., oligonucleotides including barcodes, which can be used to identify antibody type, e.g., the target specificity of an antibody. These reagents can then be used to bind to the proteins within or on cells, thereby associating the nucleic acids carried by the affinity reagents to the cells to which they are bound. These cells can then be processed through a barcoding workflow as described herein to attach barcodes to the nucleic acid labels on the affinity reagents. Techniques of library preparation, sequencing, and bioinformatics may then be used to group the sequences according to cell/discrete entity barcodes. Any suitable affinity reagent that can bind to or recognize a biological sample or portion or component thereof, such as a protein, a molecule, or complexes thereof, may be utilized in connection with these methods. The affinity reagents may be labeled with nucleic acid sequences that relates their identity, e.g., the target specificity of the antibodies, permitting their detection and quantitation using the barcoding and sequencing methods described herein. Exemplary affinity reagents can include, for example, antibodies, antibody fragments, Fabs, scFvs, peptides, drugs, etc. or combinations thereof. The affinity reagents, e.g., antibodies, can be expressed by one or more organisms or provided using a biological synthesis technique, such as phage, mRNA, or ribosome display. The affinity reagents may also be generated via chemical or biochemical means, such as by chemical linkage using N-Hydroxysuccinimide (NETS), click chemistry, or streptavidin-biotin interaction, for example. The oligo-affinity reagent conjugates can also be generated by attaching oligos to affinity reagents and hybridizing, ligating, and/or extending via polymerase, etc., additional oligos to the previously conjugated oligos. An advantage of affinity reagent labeling with nucleic acids is that it permits highly multiplexed analysis of biological samples. For example, large mixtures of antibodies or binding reagents recognizing a variety of targets in a sample can be mixed together, each labeled with its own nucleic acid sequence. This cocktail can then be reacted to the sample and subjected to a barcoding workflow as described herein to recover information about which reagents bound, their quantity, and how this varies among the different entities in the sample, such as among single cells. The above approach can be applied to a variety of molecular targets, including samples including one or more of cells, peptides, proteins, macromolecules, macromolecular complexes, etc. The sample can be subjected to conventional processing for analysis, such as fixation and permeabilization, aiding binding of the affinity reagents. To obtain highly accurate quantitation, the unique molecular identifier (UMI) techniques described herein can also be used so that affinity reagent molecules are counted accurately. This can be accomplished in a number of ways, including by synthesizing UMIs onto the labels attached to each affinity reagent before, during, or after conjugation, or by attaching the UMIs microfluidically when the reagents are used. Similar methods of generating the barcodes, for example, using combinatorial barcode techniques as applied to single cell sequencing and described herein, are applicable to the affinity reagent technique. These techniques enable the analysis of proteins and/or epitopes in a variety of biological samples to perform, for example, mapping of epitopes or post translational modifications in proteins and other entities or performing single cell proteomics. For example, using the methods described herein, it is possible to generate a library of labeled affinity reagents that detect an epitope in all proteins in the proteome of an organism, label those epitopes with the reagents, and apply the barcoding and sequencing techniques described herein to detect and accurately quantitate the labels associated with these epitopes.

Primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. Primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell.

In some implementations, solid supports, beads, and the like are coated with affinity reagents. Affinity reagents include, without limitation, antigens, antibodies or aptamers with specific binding affinity for a target molecule. The affinity reagents bind to one or more targets within the single cell entities. Affinity reagents are often detectably labeled (e.g., with a fluorophore). Affinity reagents are sometimes labeled with unique barcodes, oligonucleotide sequences, or UMI's.

In some implementations, a RT/PCR polymerase reaction and amplification reaction are performed, for example in the same reaction mixture, as an addition to the reaction mixture, or added to a portion of the reaction mixture.

In one particular implementation, a solid support contains a plurality of affinity reagents, each specific for a different target molecule but containing a common sequence to be used to identify the unique solid support. Affinity reagents that bind a specific target molecule are collectively labeled with the same oligonucleotide sequence such that affinity molecules with different binding affinities for different targets are labeled with different oligonucleotide sequences. In this way, target molecules within a single target entity are differentially labeled in these implements to determine which target entity they are from but contain a common sequence to identify them from the same solid support.

In another aspect, embodiments herein are directed at characterizing subtypes of cancerous and pre-cancerous cells at the single cell level. The methods provided herein can be used for not only characterization of these cells, but also as part of a treatment strategy based upon the subtype of cell. The methods provided herein are applicable to a wide variety of caners, including but not limited to the following: Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Astrocytomas, Atypical Teratoid/Rhabdoid Tumor, Childhood, Central Nervous System (Brain Cancer), Basal Cell Carcinoma, Bile Duct Cancer, Bladder Cancer. Childhood Bladder Cancer, Bone Cancer (includes Ewing Sarcoma and Osteosarcoma and Malignant Fibrous Histiocytoma), Brain Tumors, Breast Cancer, Childhood Breast Cancer, Bronchial Tumors, Burkitt Lymphoma (Non-Hodgkin Lymphoma, Carcinoid Tumor (Gastrointestinal), Childhood Carcinoid Tumors, Cardiac (Heart) Tumors, Central Nervous System tumors. Atypical Teratoid/Rhabdoid Tumor, Childhood (Brain Cancer), Embryonal Tumors, Childhood (Brain Cancer), Germ Cell Tumor (Childhood Brain Cancer), Primary CNS Lymphoma, Cervical Cancer, Childhood Cervical Cancer, Cholangiocarcinoma, Chordoma (Childhood), Chronic Lymphocytic Leukemia (CLL), Chronic Myelogenous Leukemia (CML), Chronic Myeloproliferative Neoplasms, Colorectal Cancer, Childhood Colorectal Cancer, Craniopharyngioma (Childhood Brain Cancer), Cutaneous T-Cell Lymphoma, Ductal Carcinoma In Situ (DCIS), Embryonal Tumors, (Childhood Brain CNS Cancers), Endometrial Cancer (Uterine Cancer), Ependymoma, Esophageal Cancer, Childhood Esophageal Cancer, Esthesioneuroblastoma (Head and Neck Cancer), Ewing Sarcoma (Bone Cancer), Extracranial Germ Cell Tumors, Extragonadal Germ Cell Tumors, Eye Cancer, Childhood Intraocular Melanoma, Intraocular Melanoma, Retinoblastoma, Fallopian Tube Cancer, Fibrous Histiocytoma of Bone (Malignant, and Osteosarcoma), Gallbladder Cancer, Gastric (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis (Langerhans Cell Cancer), Hodgkin Lymphoma, Hypopharyngeal Cancer (Head and Neck Cancer), Intraocular Melanoma, Childhood Intraocular Melanoma, Islet Cell Tumors,(Pancreatic Neuroendocrine Tumors), Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Melanoma (Intraocular Eye), Childhood Intraocular Melanoma, Merkel Cell Carcinoma (Skin Cancer), Mesothelioma, Childhood Mesothelioma, Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes—see Unusual Cancers of Childhood, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, (Acute AML), Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer (Lip and Oral Cavity Cancer and Oropharyngeal Cancer), Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer, Parathyroid Cancer, Penile Cancer, Pharyngeal Cancer, Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Stomach (Gastric) Cancer, Childhood Stomach, T-Cell Lymphoma, Testicular Cancer, Childhood Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter Kidney (Renal Cell Cancer), Ureter and Renal Pelvis (Transitional Cell Cancer Kidney Renal Cell Cancer), Urethral Cancer, Uterine Cancer (Endometrial), Uterine Sarcoma, Vaginal Cancer, Childhood Vaginal Cancer, Vascular Tumors (Soft Tissue Sarcoma), Vulvar Cancer, Wilms Tumor (and Other Childhood Kidney Tumors).

Embodiments of the invention may select target nucleic acid sequences for genes corresponding to oncogenesis, such as oncogenes, proto-oncogenes, and tumor suppressor genes. In some embodiments the analysis includes the characterization of mutations, copy number variations, and other genetic alterations associated with oncogenesis. Any known proto-oncogene, oncogene, tumor suppressor gene or gene sequence associated with oncogenesis may be a target nucleic acid that is studied and characterized alone or as part of a panel of target nucleic acid sequences. For examples, see Lodish H, Berk A, Zipursky S L, et al. Molecular Cell Biology. 4th edition. New York: W. H. Freeman; 2000. Section 24.2, Proto-Oncogenes and Tumor-Suppressor Genes. Available from: https://www.ncbi.nlm.nih.gov/books/NBK21662/, incorporated by reference herein.

As used herein, the term “panel” refers to a group of amplicons that target a specific genome of interest or target a specific loci of interest on a genome.

As used herein, the term “circuitry” may refer to, be part of, or include an Application Specific Integrated Circuit (ASIC), an electronic circuit, a processor (shared, dedicated, or group), and/or memory (shared, dedicated, or group) that execute one or more software or firmware programs, a combinational logic circuit, and/or other suitable hardware components that provide the described functionality. In some embodiments, the circuitry may be implemented in, or functions associated with the circuitry may be implemented by, one or more software or firmware modules. In some embodiments, circuitry may include logic, at least partially operable in hardware. Embodiments described herein may be implemented into a system using any suitably configured hardware and/or software.

Other aspects of the disclosure are described in reference to the following exemplary embodiments and relate to method and apparatus to design assays optimized for the desired target(s) detection. In one implementation, a Machine Learning (ML) algorithm and engine is disclosed to optimize amplicon design for uniform amplification by making reliable performance prediction.

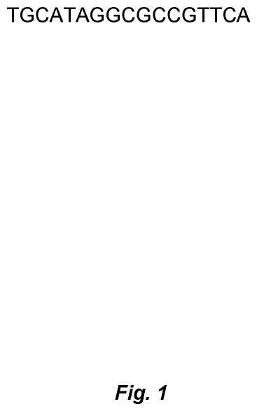

FIG. 1 is a representation of a single-stranded DNA sequence of a target molecule. Specifically, FIG. 1 illustrates a target DNA strand having 17 nucleotides. The target sequence of FIG. 1 may correspond to a mutation under study. Detection of the target DNA strand of FIG. 1, for example, may lead to detecting and identifying presence of sarcoma. To this end an assay may be designed and configured to specifically detect the presence of target DNA of FIG. 1.

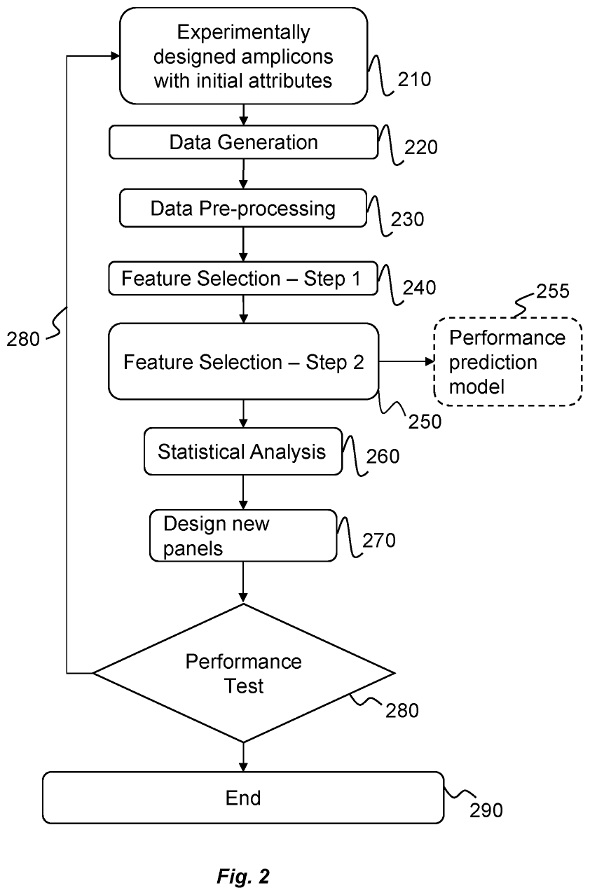

FIG. 2 illustrates an exemplary flow diagram of an overall ML training process according to one embodiment of the disclosure. The experimental design step is undertaken at step 210. Here, multiple panels with various sizes can be designed with amplicons spanning a wide range of design properties. The experimental designs can be made using conventional amplicon techniques to target gene loci of interest. The design properties may be dictated by the target detection objective. The design properties may include, among others, length, secondary structure prediction, primer specificity and amplicon GC. In one exemplary embodiment, the panel may be relatively small, for example, up to 20 amplicons to target 20 loci. In another example, the panel may be larger, for example, 180-250, or more amplicons. Each panel may have a different list of preliminary attributes or design properties. The number of initial amplicon attributes may be narrow or large depending on the desired amplicon design. The initial attributes may be selected to cover a large variety of amplicon performances.

An exemplary set of initial primer and amplicon attributes may include primer length, percentage of GC content in primer, GC content at 3′end of primer, GC content at 5′end of primer, number of G or C bases within the last five bases of 3′end, stability for the last five 3′ bases in primer (measured by maximum dG—Gibbs Free Energy—for disruption the structure), number of unknown bases in primer, number of ambiguous bases in primer, ambiguity code for ambiguous bases, long runs of single base in primer, number of tandem repeats in primer, number of dinucleotide repeats in primer, position of dinucleotide repeats in primer, number of trinucleotide repeats in primer, position of trinucleotide repeats in primer, number of tetranucleotide repeats in primer, position of tetranucleotide repeats in primer, number of pentanucleotide repeats in primer, position of pentanucleotide repeats in primer, number of hexanucleotide repeats in primer, position of hexanucleotide repeats in primer, primer melting temperature, melting temperature difference between forward and reverse primers, number of inverted repeats in primer, length of inverted repeats in primer, percentage of GC content in inverted repeats in primer, number of primer secondary hairpin structure, dG value of primer secondary hairpin structure, in-silico melting temperature of predicted primer secondary hairpin structure, primer self-dimer folding dG value, in-silico melting temperature of predicted primer self-dimer folding, primer pair heterodimer (cross dimers), primer pair heterodimer folding dG value, primer pair heterodimer melting temperature, number of primer heterodimers in a pool of primers, folding dG value for all in-silico predicted heterodimers, in-silico melting temperature of all in-silico predicted primer heterodimers, number of primer mispriming sites in template library, number of primer mispriming site in a pool of amplicons, number of primer priming sites with no mismatch in last 10 bases of 3′end, number of primer priming sites with no mismatch in last 3 bases of 3′end, number of primer priming sites with 1 mismatch in last 10 bases of 3′end, number of primer priming sites with 1 mismatch in last 3 bases of 3′end, number of primer priming sites with 1 mismatch in last 5 bases of 3′end, number of primer priming sites with 2 mismatch in last 10 bases of 3′end, number of primer priming sites with 2 mismatch in last 3 bases of 3′end, number of primer priming sites with 2 mismatch in last 10 bases of 3′end, number of primer priming sites with 2 mismatch in last 3 bases of 3′end, number of primer priming sites with 1 mismatch in last 5 bases of 3′end, number of SNP (single nucleotide polymorphisms) in primer, number of common SNP (>1%) in primer, number of one nucleotide substitution SNP in primer, position of one nucleotide substitution SNP in primer, number of one nucleotide deletion SNP in primer, position of one nucleotide deletion SNP in primer, number of one nucleotide insertion SNP in primer, position of one nucleotide insertion SNP in primer, amplicon length, percentage of GC content in amplicon, melting temperature of amplicon, insert length, percentage of GC content in insert, melting temperature of insert, percentage of GC content in first 100 bp in 5′end of amplicon, melting temperature of first 100 bp in 5′end of amplicon, percentage of GC content in last 150 bp in 3′end of amplicon, melting temperature of last 150 bp in 5′end of amplicon, target position to the 5′ end of amplicon, target position to the 3′ end of amplicon, target position to the 5′end of insert, target position to the 3′end of insert, bases of target inside forward primer, bases of target inside reverse primer, number of homopolymer runs in amplicon, length of homopolymer A runs in amplicon, position of homopolymer A in amplicon, length of homopolymer T runs in amplicon, position of homopolymer T in amplicon, length of homopolymer C runs in amplicon, position of homopolymer C in amplicon, length of homopolymer G runs in amplicon, position of homopolymer G in amplicon, number of tandem repeats in amplicon, number of dinucleotide repeats in amplicon, position of dinucleotide repeats in amplicon, number of trinucleotide repeats in amplicon, position of trinucleotide repeats in amplicon, number of tetranucleotide repeats in amplicon, position of tetranucleotide repeats in amplicon, number of pentanucleotide repeats in amplicon, position of pentanucleotide repeats in amplicon, number of hexanucleotide repeats in amplicon, position of hexanucleotide repeats in amplicon, target position to the homopolymers, target position to the tandem repeats, number of common SNP in amplicon, position of common SNP in amplicon, number of common SNP in insert, position of common SNP in insert, target position to common SNPs, insert specificity in designed genome, the minimal sequencing quality allowed for primer, the minimal sequencing quality allowed for 3′ end last five bases of primer, space between amplicons, maximum overlapping bases allowed for amplicons.

It should be noted that the design parameters provided herein are exemplary and other design parameters may be used without deviating from the disclosed principles.

Step 220 relates to data generation. Here, the experimentally designed amplicons are used to sequence a target DNA and each amplicon's performance is recorded. The sequenced DNA is then read and one or more data tables may be generated to quantify performance of each amplicon design and its attributes from step 210.

At step 230, the tested amplicons are classified into different categories depending on their performance in order to identify a plurality of primary attributes from a selected list of attributes. This step may also be called the labeling step since each tested amplicon is labeled according to its performance as measured against a standard performance threshold. Amplicon classification can be implemented in different ways. In one implementation according to an embodiment of the disclosure, a benchmark or threshold is dynamically calculated using the average performance of all tested amplicons. Each tested amplicon is then compared in different criteria against the benchmark. As a result, each amplicon is then labeled with a metric to denote its performance against the known benchmark. In an exemplary embodiment, an additional step of normalization or read-count may be performed for each amplicon. The read-count can be normalized for each amplicon as a read percentage of each cell for example by dividing the read count of one amplicon to the total number of read counts of each cell.

For example, amplicons may be labeled low-, average- and high-performers based on the respective amplicon' s normalized read value. At the end of step 230, a plurality of primary attributes are identified from a list of initial amplicon attributes which were used at step 210.

The primary attributes of each amplicon, even if labeled, may be too numerous to provide meaningful data from a myriad of tested amplicons. Thus, it is important to select key features from the primary attributes that lead to identifying significant attributes. Put differently, the primary attribute data must be analyzed to discern a select, key, set of attributes called significant attributes. The significant attributes can then be used as criteria to identify suitable and/or highly performing amplicons. Steps 240 and 250 of FIG. 2 relate to selecting the key (significant) attribute (or features) from a large list of primary attributes. Once the key features are selected, statistical data analysis may be conducted on the selected key attributes. The results of the statistical analysis can be used to design amplicons for the target sequence.

As is conventionally known, Random Forests or random decision forests are an ensemble or machine learning method for classification, regression or tasks that operate by constructing a multitude of decision trees at training time and outputting the class that is the mode of the classes (classification) or mean prediction (regression) of the individual decision trees. Random decision forests correct for decision trees habit of overfitting to their training set. Thus, according to one embodiment of the disclosure, random forest classifier can be used to calculate feature importance.

Step 240 relates to classified into categories based on their performance. That is, the design properties of the amplicons are used for classification. In an exemplary embodiment, the so-called random forest statistical algorithm is used to calculate feature importance. By way of example, recursive feature elimination (RFE) or the so-called Select-From-Model (SFM) techniques may be used to rank features based on their importance. In an exemplary application, both techniques are applied to the data and the common top features from each model is used to form the primary attribute list. The resulting selection may be, for example, 8-10 primary attributes from a list of 150-200 initial attributes.

Step 250 relates to the second feature selection step, the correlation study. Here, correlation of numeric features are studied to identify and remove highly correlated features. Only independent features may be selected for the statistical analysis step 260. The correlation study identifies highly correlated attributes and categories. Highly correlated attributes are those in which a change in one attribute causes a change in another attribute. The highly correlated attributes may be identified in the correlation study and discounted or disregarded in order to identify and select independent features. The selection of the independent features provides for a more precise selection of amplicons. In one embodiment, the selection of the independent features may reduce the number of primary attributes down to 4-8 significant attributes.

Step 255 may be performed optionally as a performance prediction model. The performance prediction model works on the performance prediction engine. Here, the selected attributes and performance labels are used to train and test performance prediction model. That is, this data is used to train different ML classification models with K-fold cross validation.

The significant attributes which were identified at step 250 (e.g., 4-8 attributes) are subjected to statistical analysis at step 260. The statistical analysis may comprise calculation of the key statistical parameters (e.g., mean, median, mode, standard deviation) for each of the significant attributes which were identified at step 250.

The above information is then used at step 270 to design new panels. That is, selected amplicons whose performance closely match the target DNA may be used to design new panels. Closely match meaning the efficient capture and sequencing of the target DNA. In one embodiment, the top features (i.e., independent, non-correlated, attributes from step 250) along with the statistical values for the top features (step 260) are used to design new panels.

The performance of the new panels may be measured at step 280 by sequencing new panels. If the new panels perform satisfactorily, the process terminates at step 290. If, on the other hand, the new panels fail to perform as desired or if additional improvement is sought, the process can revert to step 210 as shown by arrow 280. Step 280 may be optionally performed. Step 290 denotes the end of the flow diagram of FIG. 2.

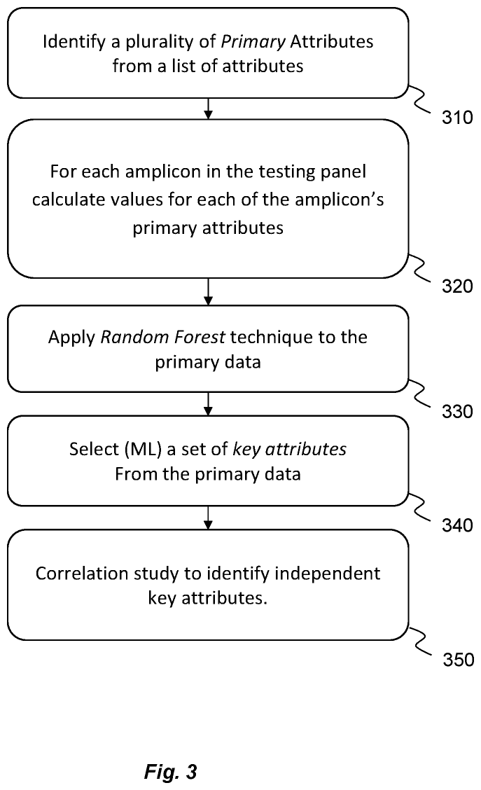

FIG. 3 illustrates an exemplary feature selection algorithm according to one embodiment of the disclosure. At step 310, a plurality of primary attributes are selected from a list of attributes.

As stated in relation to FIG. 2, the primary attributes may include any of primer length, percentage of GC content in primer, stability for the last five 3′ bases in primer), long runs of single base in primer, primer melting temperature, melting temperature difference between forward and reverse primers, number of inverted repeats in primer, length of inverted repeats in primer, number of primer secondary hairpin structure, dG value of primer secondary hairpin structure, in-silico melting temperature of predicted primer secondary hairpin structure, primer self-dimer folding dG value, in-silico melting temperature of predicted primer self-dimer folding, primer pair heterodimer, primer pair heterodimer folding dG value, primer pair heterodimer melting temperature, number of primer heterodimers in a pool of primers, folding dG value for all in-silico predicted heterodimers, in-silico melting temperature of all in-silico predicted primer heterodimers, number of primer mispriming sites in template library, number of primer mispriming site in a pool of amplicons, number of primer priming sites with no mismatch in last 10 bases of 3′end, number of primer priming sites with no mismatch in last 3 bases of 3′end, number of primer priming sites with 1 mismatch in last 10 bases of 3′end, number of primer priming sites with 1 mismatch in last 3 bases of 3′end, number of primer priming sites with 1 mismatch in last 5 bases of 3′end, number of primer priming sites with 2 mismatch in last 10 bases of 3′end, number of primer priming sites with 2 mismatch in last 3 bases of 3′end, number of primer priming sites with 2 mismatch in last 10 bases of 3′end, number of primer priming sites with 2 mismatch in last 3 bases of 3′end, number of primer priming sites with 1 mismatch in last 5 bases of 3′end, number of SNP (single nucleotide polymorphisms) in primer, number of common SNP (>1%) in primer, number of one nucleotide substitution SNP in primer, position of one nucleotide substitution SNP in primer, number of one nucleotide deletion SNP in primer, position of one nucleotide deletion SNP in primer, number of one nucleotide insertion SNP in primer, position of one nucleotide insertion SNP in primer, amplicon length, percentage of GC content in amplicon, melting temperature of amplicon, insert length, percentage of GC content in insert, melting temperature of insert, target position to the 5′ end of amplicon, target position to the 3′ end of amplicon, target position to the 5′end of insert, target position to the 3′end of insert, number of homopolymer runs in amplicon, length of homopolymer A runs in amplicon, position of homopolymer A in amplicon, length of homopolymer T runs in amplicon, position of homopolymer T in amplicon, length of homopolymer C runs in amplicon, position of homopolymer C in amplicon, length of homopolymer G runs in amplicon, position of homopolymer G in amplicon, number of tandem repeats in amplicon, number of common SNP in amplicon, position of common SNP in amplicon, number of common SNP in insert, position of common SNP in insert, target position to common SNPs, insert specificity in designed genome, the minimal sequencing quality allowed for primer, space between amplicons, maximum overlapping bases allowed for amplicons.

At step 320 an amplicon panel is tested to obtain data for each of the primary attributes for each of the amplicons in the amplicon panel. That is, for each amplicon in the testing panel, values for each primary attribute of the amplicon are calculated. An exemplary table of 600 amplicons tested against 20 primary attributes is provided at TABLE 1 below.

TABLE 1
Exemplary Primary Attribute Table

| | | | | | |
|---|---|---|---|---|---|
| | 1. Primer | | | | |
| Amp. ID | length | 2. AT % | 3. GC % | . . . | 20. Performance |

Amp. 1
Amp. 2
. . .
Amp. 599
Amp. 600

It should be noted that TABLE 1 is exemplary and non-limiting. Different primary attributes may be selected for a desired application without departing from the disclosed principles.

Referring again to FIG. 3, at step 330, the random forest technique is applied to primary data set of TABLE 1 for feature selection. As discussed in relation to FIG. 2, the design properties of the amplicons and the panels are the features or attributes.

In the feature selection step 330 the random forest classifier is used to: (1) calculate feature importance (common top features identified using two feature selection methods were selected); and (2) the numeric features were correlated to identify and remove highly correlated features to thereby arrive at significant features or attributes. These steps may be implemented independently. For example, at step 340 a set of key attributes may be selected from the primary set of attributes. Given the large volume of data (e.g., TABLE 1), ML may be used to implement step 340. At step 350, correlation study is conducted to identify independent key attributes. In one embodiment, only independent key attributes are used for statistic analysis. By way of example, applying the techniques of FIG. 3, to TABLE 1, the significant features are illustrated below at TABLE 2.

TABLE 2
Results of Correlation Study to Identify Significant Attributes

| | | |
|---|---|---|
| Amp. ID | 1. Primer length | 2. GC % |

Amp. 1
Amp. 2
. . .
Amp. 599
Amp. 600

In the exemplary embodiment of TABLE 2, only two significant attributes were selected from the twenty primary attributes. The two significant attributes of TABLE 2 were deemed to be independent and non-correlated. Steps 330 and 340 may be conducted using disclosed algorithms by one or more processors. To this end, according to one embodiment of the disclosure artificial intelligence (AI) and ML may be used to train the one or more processor to organize and correlate data to select the preferred amplicons and their characteristics.

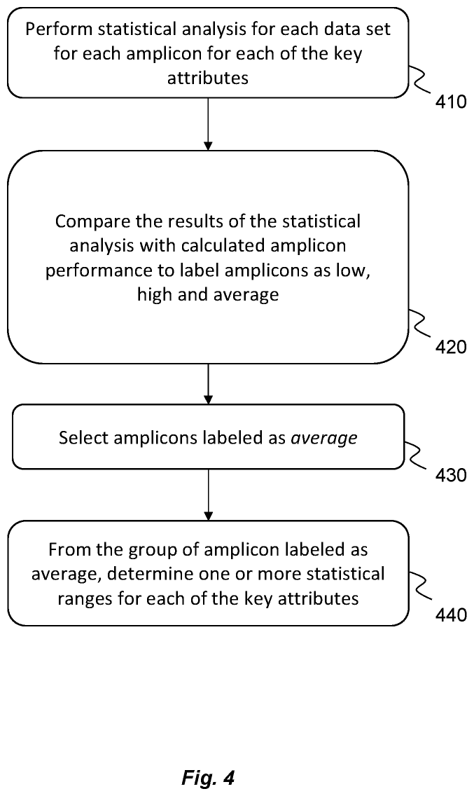

FIG. 4 is an exemplary illustration of a process flow for implementing statistical analysis and the design steps according to one embodiment of the disclosure. The flow diagram of FIG. 4 may complement step 260 of FIG. 2. In performing the steps of FIG. 4, multiple panels with various sizes may be designed with amplicons spanning a wide range of design properties. Next, the preliminary steps discussed in relation to FIGS. 2 and 3 may be applied to the results to arrive at a table of significant or key attributes.

At step 410, statistical analysis is applied to each data set for each amplicon for each of the key attributes. The statistical analysis may include, for example, determining key statistical parameters (e.g., mean, mode, median and standard deviation) for each of the dataset for each amplicon. In reference to TABLE 2, this would mean determining statistical parameters for each of the significant attributes for each amplicon.

At step 420, the statistical parameter values obtained from step 410 were compared against the existing standards to label each amplicon as low-, average- and high-performers (step 430). It should be noted that the values for the so-called existing threshold is arbitrary and may be selected as a function of empirical evidence.

In an exemplary embodiment, the amplicons that are labeled average-performers are selected while amplicons that are labeled low- or high-performers are disregarded. This is shown in step 430. It should be noted that depending on the application, amplicons that are labeled low- or high-performers may be selected without departing from the disclosed principles.

At step 440, one or more statistical ranges are calculated for each of the key attributes for each of the amplicons selected as average-performers. Based on this information, amplicon panels with key attribute values within the obtained statistical ranges may be designed for the desired application.

Due to the computational complexity of disclosed embodiments, the different algorithms of FIGS. 2-4 may be implemented with machine language (software) in a microprocessor environment (hardware). In an exemplary embodiment of the disclosure, ML can be trained to identify data trends and relationship between attributes such that correlated attributes may be identified and separated from independent attributes. Similarly, the statistical analysis may be implemented in software, hardware or a combination of software and hardware. An exemplary implementation includes instruction which may be stored at one or more memory circuitries and executed on one or more processor circuitries to implement the principles disclosed herein. The following is a brief description of such exemplary systems for implementing the disclosed principles. It should be noted that the disclosed embodiments are exemplary and non-limiting.

An exemplary embodiment of the disclosure comprises the steps of (A) data preparation, and (B) the iterative training and testing the data model. The data preparation step comprises:
(1) Providing training data table input set to form an input data set; the table comprising a plurality of amplicons with each amplicon having an identifier;
(2) providing a plurality of attributes and a performance indicators for each amplicon; and
(3) selecting a classification model (e.g., random forest) to select a key subset of attributes from among the plurality of attributes to generate a subset input data; (a table with 5-6 column and the performance column).

The iterative training and testing of the model comprises:
(1) randomly splitting the subset input data set to two groups: (a) training dataset, and (b) testing dataset;
(2) training the model on the training dataset to associate one or more feature of the subset of input data with the performance label to obtain a predictive factor;
(3) evaluating accuracy of the predictive factor using testing dataset.

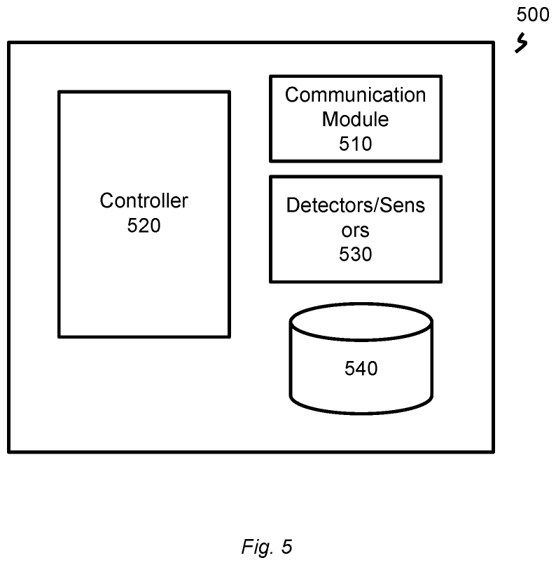

FIG. 5 shows an exemplary system for implementing an embodiment of the disclosure. In FIG. 5, system 500 may comprise hardware, software or a combination of hardware and software programmed to implement steps disclosed herein, for example, the steps of flow diagram of FIG. 5. In one embodiment, system 500 may comprise an Artificial Intelligence (AI) CPU. For example, apparatus 500 may be an ML node, an MEC node or a DC node. In one exemplary embodiment, system 500 may be implemented at an Autonomous Driving (AD) vehicle. At another exemplary embodiment, system 500 may define an ML node executed external to the vehicle.

System 500 may comprise communication module 510. The communication module may comprise hardware and software configured for landline, wireless and optical communication. For example, communication module 510 may comprise components to conduct wireless communication, including WiFi, 5G, NFC, Bluetooth, Bluetooth Low Energy (BLE) and the like. Controller 520 (interchangeably, micromodule) may comprise processing circuitry required to implement one or more steps illustrates in FIGS. 2-4. Controller 520 may include one or more processor circuitries and memory circuitries. Controller 520 may communicate with memory 540. Memory 540 may store one or more instructions to generate data tables, as described above, and to implement feature selection and statistical analysis, for example.

### Exemplary Methods

Ten (10) different panels were designed with amplicons spanning a wide range of design properties such as amplicon GC, length, secondary structure prediction, primer specificity. These panels were synthesized and processed through Tapestri® single cell DNA platform. We pre-processed the raw reads, mapped the reads, called cells and generated the amplicon-cell read matrix using the analytical pipeline. The tested amplicons were classified into one of low-performer, OK-performer and high-flyer based on their normalized reads-per-cell value.

The design properties of the amplicon are the features. Highly correlated features were identified and pruned. The random forest classifier was used to calculate feature importance. Top features were identified using two different feature selection methods. We then analyzed the range of the top features for each class and their significance of variance between classes. These ranges were then used as parameters in the assay design pipeline.

### Results

To test the performance of the design pipeline with new parameters, we designed a small (31), medium (128) and large (287) amplicon panel. Multiple runs were conducted for each panel with different cell types. We were able to achieve high panel performance of 97%, 92% and 88% across the three panels. The new parameters resulted in approximately 10-20% improvement in panel uniformity. We are working on further optimizing the performance prediction engine by using different ML classification models with K-fold cross validation, training using larger group of amplicons and optimizing features using combination of properties.

### Additional Exemplary Embodiments

The following examples are provided to further illustrate the disclosed principles. These examples are non-limiting and illustrative. It is noted that one of ordinary skill in the art may modify the examples without departing from the disclosed principles.

Example 1 is directed to a method to configure amplicons having pre-defined performance attributes, the method comprising: providing a plurality of primary amplicons targeted to one or more regions of interest of a genome, each of the plurality of amplicons having a plurality of initial attributes; sequencing each of the plurality of primary amplicons with a single cell targeted DNA panel and ranking performance of each sequenced amplicon; from among the ranked amplicons: (i) selecting a plurality of key attributes, and (ii) selecting one or more substantially independent and non-correlating attributes, to form a group of selected primary amplicon attributes; calculating a plurality of statistical parameters for each of the selected primary amplicon attributes; and configuring a plurality of secondary amplicons wherein the secondary amplicons comprise secondary amplicon parameters consistent with the statistical parameters of the selected primary amplicons.

Example 2 is directed to the method of Example 1, wherein the genome defines a single-strand DNA.

Example 3 is directed to the method of Example 2, wherein the genome defines a single-strand DNA associated with a predefined variant.

Example 4 is directed to the method of Example 1, wherein the initial attributes are selected from a group consisting of a primer length, a percentage of GC content in a primer, a GC content at 3′end of primer, a GC content at 5′end of primer and a number of G or C bases within the last five bases of 3′end of the primer.

Example 5 is directed to the method of Example 1, wherein ranking performance of each sequenced amplicon further comprises comparing performance of each sequenced amplicon in against a performance threshold.

Example 6 is directed to the method of Example 1, selecting a plurality of key attributes further comprises applying a first ranking model to identify key attributes.

Example 7 is directed to the method of Example 1, wherein the first ranking model comprises Recursive Feature Elimination (RFE).

Example 8 is directed to the method of Example 1, selecting a plurality of key attributes further comprises applying a first and a second ranking model and selecting at least one feature selected by both the first and the second models.

Example 9 is directed to the method of Example 8, wherein the first model comprises RFE and the second model comprises a weighted model.

Example 10 is directed to the method of Example 1, wherein selecting substantially independent and non-correlating attributes further comprises determining correlation between attributes and selecting attributes that are substantially void of correlation with other attributes to form a group of primary amplicon attributes.

Example 11 is directed to the method of Example 1, wherein the secondary amplicons are targeted to the one or more regions of interest.

Example 12 is directed to a non-transient machine-readable medium including instructions to configure amplicons having pre-defined performance attributes, which when executed on one or more processors, causes the one or more processors to: receive empirical data of a plurality of initial attributes from a panel of primary amplicons sequenced with target molecules, each of the initial attributes defining at least one performance criteria for a respective amplicon; rank performance of each amplicon according to a predefined criteria; from among the ranked amplicons: (i) select a plurality of key attributes, and (ii) select one or more substantially independent and non-correlating attributes, to form a group of selected primary amplicon attributes; calculate a plurality of statistical parameters for each of the selected primary amplicon attributes; and configure a plurality of secondary amplicons wherein the secondary amplicons comprise secondary amplicon parameters consistent with the statistical parameters of the selected primary amplicons.

Example 13 is directed to the medium of Example 12, wherein the genome defines a single-strand DNA.

Example 14 is directed to the medium of Example 13, wherein the genome defines a single-strand DNA associated with a predefined variant.

Example 15 is directed to the medium of Example 12, wherein the initial attributes are selected from a group consisting of a primer length, a percentage of GC content in a primer, a GC content at 3′end of primer, a GC content at 5′end of primer and a number of G or C bases within the last five bases of 3′end of the primer.

Example 16 is directed to the medium of Example 12, wherein the processor is further programmed with instructions to rank performance of each sequenced amplicon by comparing performance of each sequenced amplicon in against a standard performance threshold.

Example 17 is directed to the medium of Example 12, wherein the processor is further programmed with instructions to select a plurality of key attributes by applying a first ranking model to identify key attributes.

Example 18 is directed to the medium of Example 12, wherein the first ranking model comprises Recursive Feature Elimination (RFE).

Example 19 is directed to the medium of Example 12, wherein the processor is further programmed with instructions to select a plurality of key attributes further by applying a first and a second ranking model and by selecting at least one feature selected by both the first and the second models.

Example 20 is directed to the medium of Example 19, wherein the first model comprises RFE and the second model comprises a weighted model.

Example 21 is directed to the medium of Example 12, the processor is further programmed with instructions to select substantially independent and non-correlating attributes by determining correlation between attributes and selecting attributes that are substantially void of correlation with other attributes to form a group of primary amplicon attributes.

Example 22 is directed to the medium of Example 12, wherein the secondary amplicons are targeted to the one or more regions of interest.

The disclosed embodiments are exemplary and non-limiting. It will be evident to one of ordinary skill in the art that the disclosed principles may be applied to different samples for similar identification without departing from the instant disclosure.

What is claimed is:

1. A method to configure amplicons having pre-defined performance attributes, the method comprising:
providing a plurality of primary amplicons targeted to one or more regions of interest of a genome, each of the plurality of amplicons having a plurality of initial attributes;
sequencing each of the plurality of primary amplicons with a single cell targeted DNA panel and ranking performance of each sequenced amplicon;
from among the ranked amplicons:
(i) selecting a plurality of key attributes, and
(ii) selecting one or more substantially independent and non-correlating attributes, to form a group of selected primary amplicon attributes;
calculating a plurality of statistical parameters for each of the selected primary amplicon attributes; and
configuring a plurality of secondary amplicons wherein the secondary amplicons comprise secondary amplicon parameters consistent with the statistical parameters of the selected primary amplicons.

2. The method of claim 1, wherein the genome defines a single-strand DNA.

3. The method of claim 2, wherein the genome defines a single-strand DNA associated with a predefined variant.

4. The method of claim 1, wherein the initial attributes are selected from a group consisting of a primer length, a percentage of GC content in a primer, a GC content at 3′end of primer, a GC content at 5′end of primer and a number of G or C bases within the last five bases of 3′end of the primer.

5. The method of claim 1, wherein ranking performance of each sequenced amplicon further comprises comparing performance of each sequenced amplicon in against a performance threshold.

6. The method of claim 1, selecting a plurality of key attributes further comprises applying a first ranking model to identify key attributes.

7. The method of claim 1, wherein the first ranking model comprises Recursive Feature Elimination (RFE).

8. The method of claim 1, selecting a plurality of key attributes further comprises applying a first and a second ranking model and selecting at least one feature selected by both the first and the second models.

9. The method of claim 8, wherein the first model comprises RFE and the second model comprises a weighted model.

10. The method of claim 1, wherein selecting substantially independent and non-correlating attributes further comprises determining correlation between attributes and selecting attributes that are substantially void of correlation with other attributes to form a group of primary amplicon attributes.

11. The method of claim 1, wherein the secondary amplicons are targeted to the one or more regions of interest.

12. A non-transient machine-readable medium including instructions to configure amplicons having pre-defined performance attributes, which when executed on one or more processors, causes the one or more processors to:
receive empirical data of a plurality of initial attributes from a panel of primary amplicons sequenced with target molecules, each of the initial attributes defining at least one performance criteria for a respective amplicon;
rank performance of each amplicon according to a predefined criteria;
from among the ranked amplicons:
(i) select a plurality of key attributes, and
(ii) select one or more substantially independent and non-correlating attributes, to form a group of selected primary amplicon attributes;
calculate a plurality of statistical parameters for each of the selected primary amplicon attributes; and
configure a plurality of secondary amplicons wherein the secondary amplicons comprise secondary amplicon parameters consistent with the statistical parameters of the selected primary amplicons.

13. The medium of claim 12, wherein the genome defines a single-strand DNA.

14. The medium of claim 13, wherein the genome defines a single-strand DNA associated with a predefined variant.

15. The medium of claim 12, wherein the initial attributes are selected from a group consisting of a primer length, a percentage of GC content in a primer, a GC content at 3′end of primer, a GC content at 5′end of primer and a number of G or C bases within the last five bases of 3′end of the primer.

16. The medium of claim 12, wherein the processor is further programmed with instructions to rank performance of each sequenced amplicon by comparing performance of each sequenced amplicon in against a standard performance threshold.

17. The medium of claim 12, wherein the processor is further programmed with instructions to select a plurality of key attributes by applying a first ranking model to identify key attributes.

18. The medium of claim 12, wherein the first ranking model comprises Recursive Feature Elimination (RFE).

19. The medium of claim 12, wherein the processor is further programmed with instructions to select a plurality of key attributes further by applying a first and a second ranking model and by selecting at least one feature selected by both the first and the second models.

20. The medium of claim 19, wherein the first model comprises RFE and the second model comprises a weighted model.

21. The medium of claim 12, the processor is further programmed with instructions to select substantially independent and non-correlating attributes by determining correlation between attributes and selecting attributes that are substantially void of correlation with other attributes to form a group of primary amplicon attributes.

22. The medium of claim 12, wherein the secondary amplicons are targeted to the one or more regions of interest.
